(19) United States
(12) Patent Application Publication
(10) Pub. No.: US 2010/0179439 A1
(43) Pub. Date: Jul. 15, 2010

(54) DEVICE AND METHOD FOR CORRECTING A BLOOD PRESSURE MEASURED

(75) Inventors: Christian Kuschel, Nuernberg (DE); Hans-Joachim Moersdorf, Fuerth (DE); Stefan Aschenbrenner, Eckental (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(73) Assignee: FRAUNHOFER-GESEWANDTEN ZUR FÖRDERUNG DER ANGEWANDT, Munich (DE)

(21) Appl. No.: 12/602,812

(22) PCT Filed: Jun. 5, 2008
(86) PCT No.: PCT/EP2008/004497

§ 371 Date: Feb. 11, 2010

(30) Foreign Application Priority Data
Jun. 6, 2007 (DE) 10 2007 026 402.1

(51) Int. Cl. A61B 5/02

(52) U.S. Cl. 600/485

(57) ABSTRACT

A device for altitude correction of a blood pressure measured at a measuring position of a living being has a transmitter, at least three receivers and an evaluating unit. The transmitter emits a signal from close to a measuring position and the at least three receivers receive the signal, wherein the receivers may be mounted to positions at different altitudes of the living being. The evaluating unit corrects the blood pressure measured on the basis of run time or phase differences of the signals received at the at least three receivers.

### BACKGROUND OF THE INVENTION

The present invention relates to a device and a method for correcting a blood pressure measured at a measuring position and, in particular, to determining the arm altitude to improve the measuring results in peripheral blood pressure measurements of a human being.

The blood pressure measured of a human being, but also of an animal, among other things, depends on a measuring position. Due to gravity, the blood pressure in a foot, for example, naturally is higher than in a head region—at least when standing upright. In order to avoid such errors caused by gravity, a blood pressure measurement is typically performed at the same altitude as the heart. However, the blood pressure measurement may also be performed at different positions of the body, as long as the error resulting as a consequence of the deviating altitude relative to the heart is compensated. When measuring the blood pressure at a wrist, for example, as is the case in commercially available apparatuses, the altitude of the hand above the heart is an essential factor influencing the blood pressure measured. If the blood pressure measured is to be freed of the error caused by the “incorrect measuring altitude”, what will be necessitated is the easiest determination possible of the altitude of the measuring arrangement above the heart.

### SUMMARY

According to an embodiment, a device for altitude correction of a blood pressure measured at a measuring position of a living being may have: a transmitter for emitting a signal from close to a measuring position; at least three receivers for receiving the signal, wherein the receivers may be mounted to positions at different altitudes of the living being; and an evaluating unit for correcting the blood pressure measured on the basis of run time or phase differences of the signals received at the at least three receivers.

According to another embodiment, a method for altitude correction of a blood pressure measured at a measuring position of a living being may have the steps of: transmitting a signal by a transmitter from close to a measuring position; receiving the signal by at least three receivers, wherein the at least three receivers are mounted to positions at different altitudes of the living being; and correcting the blood pressure measured on the basis of run time or phase differences of the signal received at the at least three receivers.

The present invention is based on the finding that a device for determining an altitude of a measuring apparatus relative to a reference point can be provided when the measuring apparatus comprises a transmitter or a transmitter is located near the measuring apparatus and emits a signal, like, for example, in the form of an electromagnetic wave of constant frequency, and the signal is received from at least three receivers. The three receivers transmit a receive signal to an evaluating unit, so that a run time or phase difference of the signals received at two of the three receivers can be determined in the evaluating unit. The run time or phase difference in turn determines a path difference between paths which the signal has traveled to the first one of the two transmitters and to the second one of the two transmitters. Similarly, another path difference can be determined during propagation to a third one of the three transmitters and the second or first one of the three transmitters.

The receive signals of the three receivers may exemplarily be electrical alternating signals the phases of which are in a fixed relation to the phases of the waves (or signals) received by the receivers. In this case, phase differences of the electrical alternating signals can be determined easily by means of a phase discriminator and the evaluating unit can determine a path length difference or path differences from it using the frequency and propagation speed. It is also possible for the phase information of the wave received to be detected and passed on to the evaluating unit in another way (like, for example, digitally).

If the three receivers are located at the body at different altitudes, advantageously along a vertical line (i.e. advantageously perpendicular), the relative altitude of the transmitter relative to the three receivers can be determined using the path differences (i.e. the run time or phase differences). Assuming the three receivers to be located in a predetermined or known distance to the heart, an altitude deviation of the transmitter relative to the heart can finally be determined from it. The altitude deviation determined may then be used to determine a correction value by which the blood pressure measurement is corrected. If the transmitter is, according to embodiments of the present invention, located as close to the measuring apparatus as possible, the most precise determination possible of the altitude deviation may be performed.

Thus, the present invention describes a device for correcting a blood pressure measured at a measuring position at a living being (like, for example, a human being), the device comprising a transmitter, at least three receivers and an evaluating unit. The transmitter emits a signal from close to the measuring position and the three receivers receive the signal, wherein the three receivers may be mounted to positions at different altitudes of the living being. The evaluating unit is configured to perform correction of the blood pressure measured on the basis of run time or phase differences of the signals arriving at the three receivers.

In embodiments, the measuring arrangement comprises a transmitter mounted to a wrist and three receivers mounted to the torso of, for example, a human being-like to the shoulder and waist. The transmitter emits an electromagnetic wave which is detected by the three receivers. Due to the relative position of the transmitter to the three receivers, differences in phase expressing themselves in phase differences result in the receivers. These phase differences can be detected and measured by phase discriminators. Using the known propagation speed (speed of light for an electromagnetic wave), the phase differences are converted into fractions of wavelengths by which the distance differences differ from one another.

The frequency of the electromagnetic wave here may be selected such that there will never be several complete wave trains in one region to be detected (like, for example, double an arm's length), since otherwise the phase angle or phase angle difference cannot be determined unambiguously and thus, the position or altitude deviation cannot be determined unambiguously. Double an arm's length, for example, here refers to the two extreme values where the blood pressure measuring apparatus or the transmitter is located at the wrist and the hand can be stretched out perpendicularly downwards on the one hand and perpendicularly upwards on the other hand. In order to ensure unambiguous measurements, a lower limit for the wavelength used (=minimum wavelength) of the electromagnetic wave should be taken into account.

On the other hand, the wavelength of the electromagnetic wave used, however, should not be selected to be too large (or the frequency should not be selected to be too small) in order for the phase angles to differ, at least two of the three receivers, to an extent allowing unambiguous detection of the phase difference. Thus, the wavelength is below a maximum wavelength. With very large wavelengths, the amplitude value of the electromagnetic wave received at the three receivers will only differ marginally, so that this marginal difference might be below a measuring threshold (measuring tolerance, like, for example, an amplitude value differing by less than five %). Since a perpendicular positioning of the three receivers along the body in itself already represents a source of error, it is of advantage to select the wavelength range of the electromagnetic wave such that receivers are allowed to detect the phase differences easily. In other words, such that the phase difference detected (or the respective difference in length) differs considerably (like, for example, by more than 20 percent) from the error resulting from deviations from an ideal perpendicular orientation of the receivers. Exemplarily, the wavelength may be in a range between 10 cm and 200 cm or in a range between 40 cm and 120 cm.

In further embodiments, the transmitter is integrated directly in the blood pressure measuring apparatus. Additionally, it is possible to determine the distance between the three receivers such that the transmitter is brought close to one of the three receivers for being calibrated, so that the phase differences of the signals received at the other ones of the three receivers are a direct measure of the distance (or altitude difference) of the other receivers to the one of the three receivers. Alternatively, the distance of the three receivers may also be measured by means of other conventional procedures.

In other embodiments, the device comprises further receivers so that the measuring precision can be increased by means of averaging. Since determining the altitude using four receivers is already over-determined, the fourth and/or any further receiver may be used for determining an error rate. With four or more receivers, the error rate determined may also be used for performing optimization with regard to the wavelength of the electromagnetic waves in order to achieve an optimum wavelength range, which, for example, allows unambiguous measurement with minimal errors, by altering the wavelength of the transmitter in this way. Additionally, it is possible, when using pulsed signals, to perform run time measurements of the pulses and thus determine path differences directly from run time differences. Both single distances and double distances (there and back) using signals reflected at the receivers may be employed for this.

The present invention is of advantage in that it offers a simple and effective and, in addition, cheap way of performing altitude correction of blood pressure measurements using simple standard components. The inventive device and the inventive method may be employed flexibly and dynamic adjustment of the blood pressure measuring value measured can be performed even with a momentary change in the relative altitude (like, for example, relative to the heart).

Other elements, features, steps, characteristics and advantages of the present invention will become more apparent from the following detailed description of the preferred embodiments with reference to the attached drawings.

### BRIEF DESCRIPTION OF THE DRAWINGS

Embodiments of the present invention will be detailed subsequently referring to the appended drawings, in which:

FIG. 1 shows a schematic illustration of an embodiment of the present invention;

FIG. 2 shows an illustration of the geometrical quantities for determining an altitude deviation;

FIG. 3a shows an illustration for optimizing a wavelength of the electromagnetic wave; and

FIG. 3b shows an illustration of an unfavorably selected wavelength of the electromagnetic wave.

### DETAILED DESCRIPTION OF THE INVENTION

Before the present invention will be detailed subsequently referring to the drawings, it is pointed out that same elements in the figures are provided with same or similar reference numerals and that a repeated description of these elements is omitted.

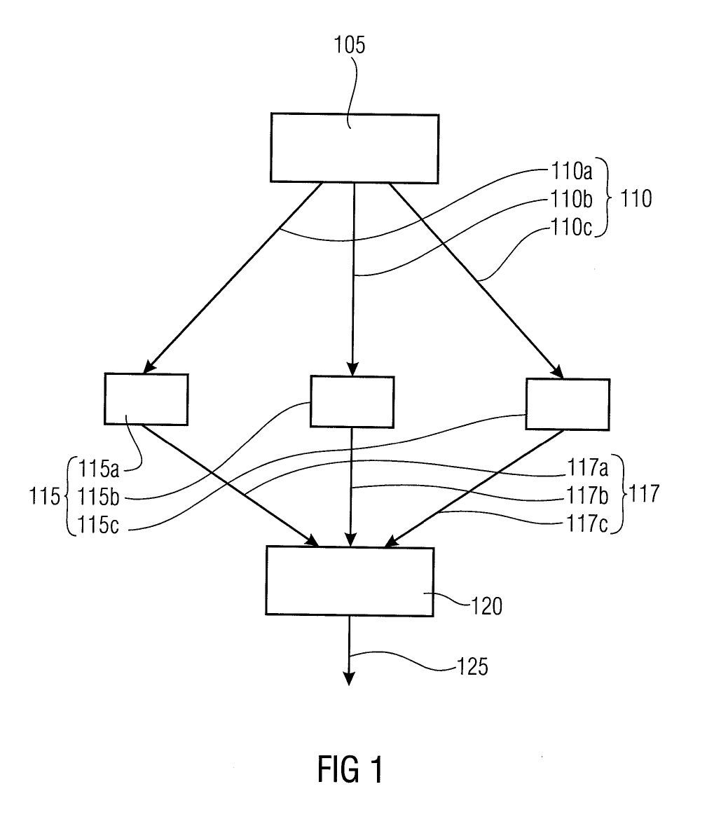

FIG. 1 shows a schematic illustration of an embodiment of the present invention. A transmitter 105 emits an electromagnetic wave 110 of a predetermined wavelength. A first part of the electromagnetic wave 110a reaches a first receiver 115a, a second part of the electromagnetic wave 110b reaches a second receiver 115b and a third part of the electromagnetic wave 110c reaches a third receiver 115c. The first receiver 115a detects the first part of the electromagnetic wave 110a and communicates a first receive signal 117a to an evaluating unit. The second receiver 115b detects the second part of the electromagnetic wave 110b and communicates a second receive signal 117b to the evaluating unit 120. The third receiver 115c detects the third part of the electromagnetic wave 110c and communicates a third receive signal 117c to the evaluating unit 120. The first, second and third receive signals 117a, 117b and 117c exemplarily contain information on the phase of the electromagnetic wave 110 or phase of the first part of the electromagnetic wave 110a at the time of receiving by the first receiver 115a. Similarly, this second receive signal 117b contains phase information of the second part of the electromagnetic wave 110b and the third receive signal 117c contains phase information regarding the third part of the electromagnetic wave 110c (like, for example, a phase value at the time of reception). If the transmitter uses pulsed signals, alternatively the three receive signals 117a, 117b and 117c may also contain information on the time of receiving the pulsed signals by the first, second and third receivers 115a, 115b and 115c.

The evaluating unit 120 compares the first receive signal 117a, the second receive signal 117b and the third receive signal 117c. Exemplarily, the evaluating unit 120 may determine a phase difference of the first part of the electromagnetic wave 110a compared to the second part of the electromagnetic wave 110b at the time of receiving the first part of the electromagnetic wave 110a by the first receiver 115a and the second part of the electromagnetic wave 110b by the second receiver 115b. The evaluating unit 120 can determine a length difference between the lengths the first part or the second part of the electromagnetic wave 110a or 110b have traveled from this phase difference. Similarly, the evaluating unit 120 can find out a phase difference between the second part of the electromagnetic wave 110b and the third part of the electromagnetic wave 110c at the time of receiving by the second receiver 115b and the third receiver 115c and determine a length difference from it. Alternatively, when using a pulsed transmitter, the time differences when receiving can be converted to length differences.

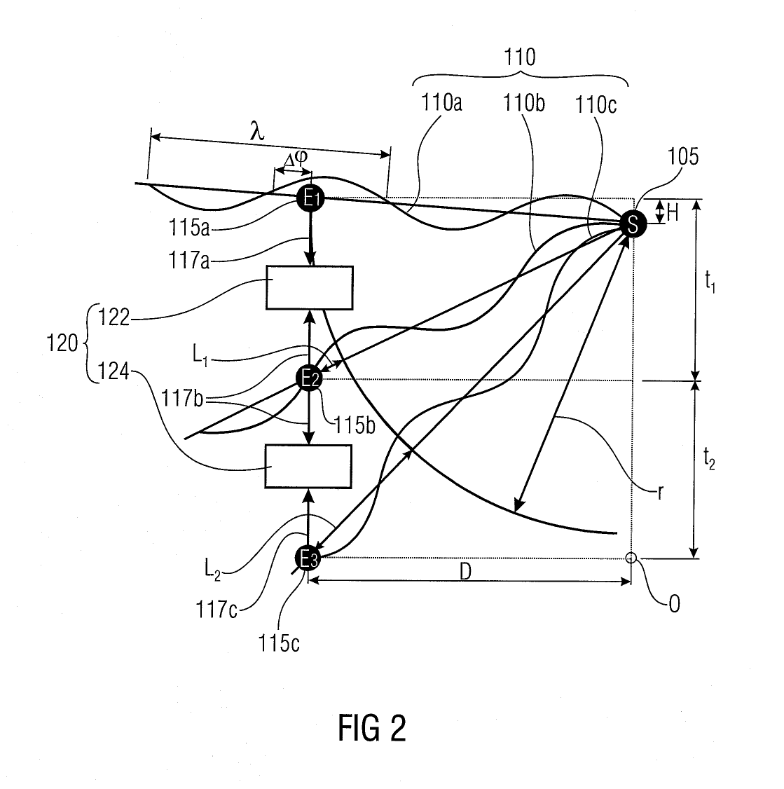

From the length differences determined therefrom (further details will be described in FIG. 2) and from the altitude differences between the first, second and third receivers 115a, 115b and 115c, the evaluating unit can determine an altitude deviation 125 where the transmitter 105 is located relative to one of the three receivers 115a, 115b, and 115c. The altitude deviation 125 may then be used to correct a blood pressure measured correspondingly.

FIG. 2 shows an illustration of the geometrical quantities for determining the altitude deviation 125, which subsequently will be referred to by H. The three receivers 115a, 115b and 115c in this illustration are arranged to be perpendicular one above the other, the third receiver 115c being located on a basic line in a distance D from a coordinate origin O. In a distance t₂, the second receiver 115b is located perpendicular above the third receiver 115c and the first receiver 115a is located in a distance t₁ perpendicular above the second receiver 115b. The coordinate origin O is selected such that the transmitter 105 is arranged to be perpendicular above the coordinate origin O. The transmitter 105 in FIG. 2 is referred to by S, the first receiver 115a is characterized by E₁, the second receiver 115b by E₂ and the third receiver 115c by E₃. The first receiver 115a is in a radial distance r from the transmitter 105, the second receiver 115b is in a second radial distance r+L₁ from the transmitter 105 and the third receiver 115c is in a third radial distance r+L₂ from the transmitter 105.

The electromagnetic wave 110 transmitted by the transmitter 105 has a wavelength λ and, at the time of receiving by the first receiver 115a, a phase value of π-Δφ (angles will subsequently be indicated in a circular measure). Since the distance of the first transmitter 105 to the second receiver 115b is greater by the first length L₁ than the distance of the first receiver 115a to the transmitter 105, the phase value of the electromagnetic wave 110b received has changed correspondingly at the second receiver 115b. Similarly, the phase value of the third part of the electromagnetic wave 110c, at the time of receiving by the third receiver 115c, has shifted correspondingly as a consequence of the distance of the third receiver 115c from the transmitter 105 increased by the third path length L₂.

The position of the point S (transmitter 105) relative to points E₁ (first receiver 115a), E₂ (second receiver 115b) and E₃ (third receiver 115c) can be determined using trigonometric laws and, using these, the (relative) altitude H of the point S (exemplarily the wrist) relative to E₁ (exemplarily the shoulder) can be determined. Determining the position of the point S takes place in R² (i.e. within a plane) with a precision of two possible positions—left and right or in mirror symmetry relative to the perpendicular straight between E₁ and E₃. Considered from the space R³, this means that determining the position takes place with a precision of a circular path with a central point on the straight E₁E₂. The position along the circle, however, remains undetermined—only the position of the circle in R³ is determined. Since, however, only the altitude H is of interest for the application aimed at, the resolution of the position which may be achieved is sufficient.

In detail, a system of equations made of three equations and three unknown quantities is solved for determining the relative altitude H (like, for example, of the wrist above or below the altitude of E₁). As can be taken from FIG. 2, the following variables are used here:

E̅₁̅E̅₂̅ be t₁,

E̅₂̅E̅₃̅ be t₂,

S̅E̅₁̅ be r, and

D be the perpendicular distance from S to the straight E₁E₂

Using this definition, the following relations result:

r²=H²+D², (1)

(r+L₁)²=(t₁−H)²+D², (2)

(r+L₂)²=(t₂+t₁−H)²+D². (3)

By substituting D²=r²−H² from (1) in (2) and (3), the following equations result:

0=t₁²−2·H·t₁−2·r·L₁−L₁², (4)

0=(t₁+t₂)²−2·H·(t₁+t₂)−2·r·l₂−L₂². (5)

Solving (5) for r and substituting in (4) will have the following result:

$$H = \frac{{{L}_{1} ( {t}_{1} + {t}_{2} )}^{2} - {L}_{2} ( {t}_{1}^{2} - {L}_{1}^{2} ) - {L}_{1} \cdot {L}_{2}^{2}}{2 \cdot ( ( {t}_{1} + {t}_{2} ) \cdot {L}_{1} - {t}_{1} \cdot {L}_{2} )} \qquad ( 6 )$$

The first receiver 115a and the second receiver 115b in this embodiment communicate the receive signals 117a, 117b to the evaluating unit 120 comprising a first phase discriminator 122 which in turn determines a first phase difference Δφ₁ between the phase of the first part of the electromagnetic wave 110a detected by the first receiver 115a and the phase of the second part of the electromagnetic wave 110b detected by the second receiver 115b. In the same manner, the second receiver 115b and the third receiver 115c transmit the receive signals 117b, 117c to a second phase discriminator 124 determining a second phase difference Δφ₂ between the phases of the electromagnetic wave detected at the second receiver 115b and the electromagnetic wave detected at the third receiver 115c. The first phase difference Δφ₁ determined at the first phase discriminator and the second phase difference Δφ₂ detected at the second phase discriminator 124 can be converted to the first length different L₁ and the second length difference L₂ using the following relations:

$$\begin{matrix}{L}_{1} = \frac{Δ {ϕ}_{1}}{2 π} λ , & ( 7 ) \\ {L}_{2} = \frac{Δ {ϕ}_{2}}{2 π} λ & ( 8 )\end{matrix}$$

wherein, as has been mentioned, angle measurement is performed in circular measure, i.e. the phase φ is periodic in 2π.

Thus, in the arrangement as is illustrated in FIG. 2, two measurements are performed, one measurement for determining the first phase difference Δφ₁ which in turn establishes the first length difference L₁, and a second measurement determining the second phase difference Δφ₂ which in turn establishes the second length difference L₂. Since the relative position of the receivers Eᵢ and thus the quantities t₁ and t₂ are known, wherein for example a certain receiver (like, for example, the second receiver E₂) may be mounted close to the heart, it is possible to determine the relative position of the point S to the certain receiver.

When optionally adding further receivers and, consequently, performing further measurements, in addition to equations (1) to (3), a fourth equation would be added and thus the system of equations would be over-determined (four equations for three unknown quantities D, r and H; tᵢ are assumed to be known or are measured)—however, the further measurement could serve as test measurement for determining an error rate, for example as a consequence of non-ideally perpendicularly oriented receivers Eᵢ (i counts the number of receivers, like, for example, i=1, 2, 3, 4) or as a consequence of an unfavorably selected wavelength λ of the transmitter 105. Determining the error rate here can take place such that three different ones of the four (or more) receivers are selected to determine different altitude deviations Hᵢ so that scattering (exemplarily expressed by standard deviation) represents a measure of the error rate. Thus, both the geometrical arrangement (orientation of receivers) and the wavelength λ selected could be optimized.

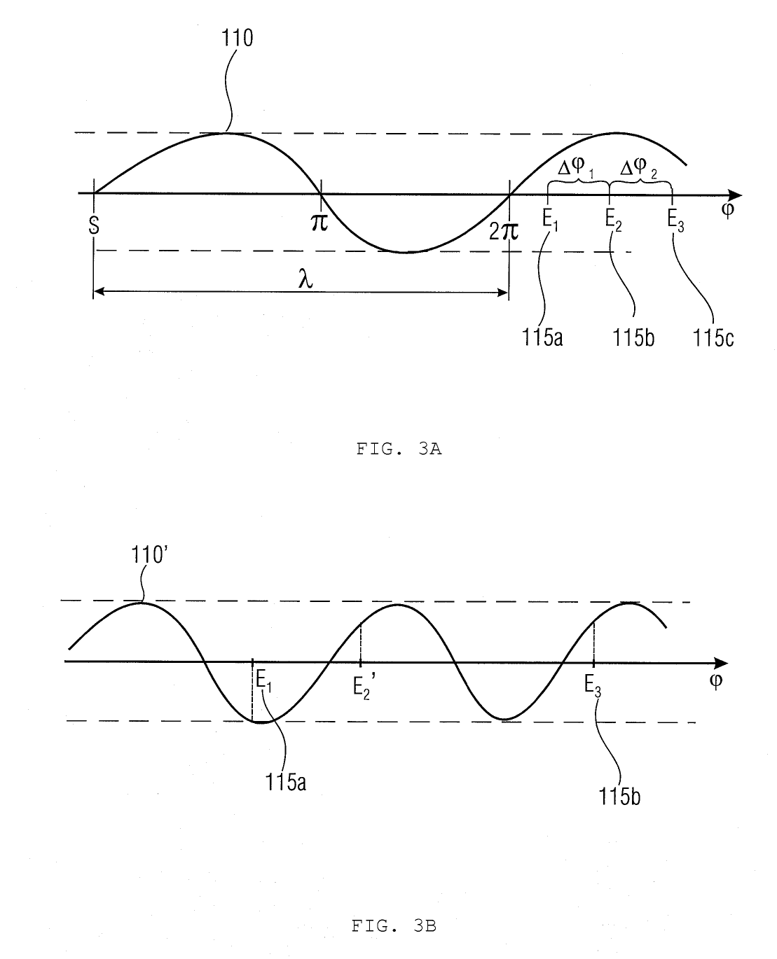

FIG. 3a shows an illustration where the three receivers, the first receiver E₁, the second receiver E₂ and the third receiver E₃, are represented along a direction so that the different distances to the point S manifest themselves in different phase values φ for the electromagnetic wave 110 emitted by the transmitter S. A phase φ=E₁ thus corresponds to a first phase value which the electromagnetic wave 110 has when received by the first receiver 115a and the phase φ=E₂ corresponds to a second phase value which the electromagnetic wave 110 has at the time of receiving by the second receiver 115b and the phase φ=E₃ corresponds to a third phase value which the electromagnetic wave 110 has at the time of receiving by the third receiver 115c. Accordingly, the first phase difference Δφ₁=E₂−E₁ in accordance with equation (7) corresponds to the first length difference L₁. In the same way, the second phase difference Δφ₁=E₃−E₂ corresponds to the second length difference L₂, in accordance with the above equation (7).

As can be seen in FIG. 3a, the electromagnetic wave 110 varies between two maximum values represented by broken lines and the wave length here is selected such that the three receivers (E₁, E₂, E₃) are within one period of the wave 110 and the amplitude of the electromagnetic wave 110 changes considerably between the first receiver 115a and the third receiver 115c. The selection of the wavelength λ of the electromagnetic wave 110 as it is shown in FIG. 3a, thus corresponds to the criteria mentioned before that there cannot be several wave periods between the receivers—the wavelength λ is both above the minimum and below the maximum wavelengths.

FIG. 3b in contrast shows another wave 110′ comprising a considerably shorter wavelength (in comparison to the distance of the receivers E₁ and E₂) so that in this case one complete period of the further wave 110′ is between the first receiver 115a and the second receiver 115b. The evaluating unit examining or determining the phase difference of the further wave 110′ at point E₁ to point E₂ cannot differentiate between point E₂ and point E₂′. Consequently, an unambiguous distance determination of the length L₁ based on this phase difference is not possible. The same would apply if the wavelength λ were selected to be so great that the amplitude between point E₁ and point E₂ only changed marginally so that, within an error tolerance, the length for the length difference L₁ cannot be determined.

Determining the phase differences Δφ₁ and Δφ₂ thus corresponds to determining run time differences of the electromagnetic wave from the transmitter 105 to the receivers 115a, 115b and 115c and, alternatively, could also take place using time measurement, like, for example, using the pulsed signals mentioned before. However, it is of advantage in the embodiment shown in FIG. 2 that no time synchronization is necessitated and the transmitter 105 can continuously transmit an electromagnetic wave 110.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1-15. (canceled)

16. A device for altitude correction of a blood pressure measured at a measuring position of a living being, comprising:
a transmitter for emitting a signal from close to a measuring position;
at least three receivers for receiving the signal, wherein the receivers may be mounted to positions at different altitudes of the living being; and
an evaluating unit for correcting the blood pressure measured on the basis of run time or phase differences of the signals received at the at least three receivers.

17. The device in accordance with claim 16, wherein the evaluating unit is configured to determine an altitude difference of the transmitter to a first receiver from the run time or phase differences.

18. The device in accordance with claim 16, wherein the signal comprises an electromagnetic wave of predetermined wave length, and wherein the at least three receivers are configured to communicate receive signals to the evaluating unit.

19. The device in accordance with claim 18, wherein the receivers detect phases of the electromagnetic wave received and the receive signals comprise information on phase values received, and wherein the evaluating unit comprises:
a first phase discriminator for determining a first phase difference Δφ1 between a second phase value received and a first phase value received; and
a second phase discriminator for determining a second phase difference Δφ2 between a third phase value received and the second phase value received.

20. The device in accordance with claim 18, wherein the predetermined wavelength corresponds to at least double an arm's length of a human being.

21. The device in accordance with claim 18, wherein the predetermined wavelength is below a maximum wavelength and the maximum wavelength is given by an amplitude value of the electromagnetic wave changing by less than 5% between the at least three receivers.

22. The device in accordance with claim 18, wherein the predetermined wavelength is in a range between 10 cm and 200 cm or in a range between 40 cm and 120 cm.

23. The device in accordance with claim 16, wherein the evaluating unit is configured to determine a relative altitude difference between the at least three receivers by the transmitter being positionable close to one of the three receivers and the relative altitude differences of the three receivers being determinable on the basis of run time or phase differences.

24. The device in accordance with claim 16, wherein the transmitter is attached to a blood pressure measuring apparatus.

25. The device in accordance with claim 16, wherein the transmitter may be mounted to a wrist.

26. The device in accordance with claim 16, comprising a first receiver, a second receiver and a third receiver, wherein the first, second and third receivers may be mounted to the human body such that the first receiver may be mounted at the altitude of a shoulder, the second receiver at the altitude of the heart and the third receiver at the altitude of the waist.

27. The device in accordance with claim 16, further comprising a fourth receiver, the fourth receiver being configured to receive the signal and the evaluating unit being configured to use the signal received of the fourth receiver as a test measurement for determining an error rate.

28. A method for altitude correction of a blood pressure measured at a measuring position of a living being, comprising:
transmitting a signal by a transmitter from close to a measuring position;
receiving the signal by at least three receivers, wherein the at least three receivers are mounted to positions at different altitudes of the living being; and
correcting the blood pressure measured on the basis of run time or phase differences of the signal received at the at least three receivers.

29. The method in accordance with claim 28, further comprising measuring relative altitude differences.

30. The method in accordance with claim 28, further comprising positioning the transmitter at a second receiver and determining relative altitude differences from the run time or phase differences.
